(19) United States
(12) Patent Application Publication
(10) Pub. No.: US 2005/0089499 A1
(43) Pub. Date: Apr. 28, 2005

(54) Active substances for use in cosmetic and/or pharmaceutical products, obtainable from the fermentation of plant components and/or plant extracts

(76) Inventors: Philippe Moussou, Nancy (FR); Louis Danoux, Sauixures Les Nancy (FR); Gilles Pauly, Nancy (FR)

Correspondence Address: COGNIS CORPORATION;PATENT DEPARTMENT, 300 BROOKSIDE AVENUE, AMBLER, PA, 19002, US

(21) Appl. No.: 10/501,623

(22) PCT Filed: Jan. 7, 2003
(86) PCT No.: PCT/EP03/00066

(30) Foreign Application Priority Data
Jan. 15, 2002 (FR) 02/00423

(51) Int. Cl. A61K 7/06; A61K 35/78

(52) U.S. Cl. 424/74; 424/750; 424/773; 424/757; 424/735; 424/727

(57) ABSTRACT

Processes for producing cosmetic and/or pharmaceutical active components which comprise: (a) providing a fermentation broth comprising a plant component selected from the group consisting of plant constituents, plant extracts and mixtures thereof; (b) inoculating the fermentation broth with a microorganism; and (c) fermenting the microorganism-containing fermentation broth to produce an active component; are described along with cosmetic and/or pharmaceutical preparations containing such active components and methods of using the same to treat the skin and/or hair.

### FIELD OF THE INVENTION

This invention relates generally to the cosmetics field and, more particularly, to new active components from fermented plant constituents and/or plant extracts, to a process for their production, to preparations containing these active components and to a number of uses for the new active components.

### PRIOR ART

The desire for eternal youth and beauty existed even in ancient times. Whereas legend has it that Cleopatra regularly bathed in asses' milk—today we know about the effect of the proteins present in such milk—less well-off ladies had to hope that their wish would be heard by the gods. It has to be assumed that this was only rarely crowned with success. Nowadays, a youthful appearance and a skin virtually free from wrinkles is not the privilege of just a few, but is basically available to all women despite the occasionally considerable differences in the price of the preparations. Even if cosmetic chemistry cannot work miracles, knowledge of the biochemical processes in the cells of skin and hair has increased enormously in recent years. As a result, there are of course theories as to how damage caused by natural ageing or environmental influences can be prevented or eliminated. However, the demands that female (and increasingly male) consumers expect such anti-ageing preparations to satisfy have also increased. Quite apart from the fact that, basically, the preparations are expected to have a “caring” character, to protect the skin from drying out and to show optimal compatibility with the skin and, optionally, the mucosa, they are required to provide protection against UV radiation and environmental toxins, to stimulate the immune system and to have anti-inflammatory activity.

In this connection, it is pointed out that the use of fermentation products of milk proteins such as, for example, kefir, kumiss, kuban, leben and mazun is well-known in the field of human nutrition [cf. for example Hesseltine, Mycologia 57, 1-148 (1965)]. Fermentation products of the agave are known as pulque. Tibi or ginger ale is obtained by fermentation of sucrose, raisins or lemons. Busa is a drink obtained by fermentation of rice and sugar. Hitherto, however, little has been known of the use of fermentation products in cosmetics. It is known that fermented whey is supposed to improve the appearance of the skin [FR-B1 2718752, World Trust Investment]. The use of kefir for treating the skin and as an agent against eczema, mycoses and acne is known from EP-A2 0315541 (L'Oréal). ES-B1 2116201 (Javier Uruena Mendez) proposes using kefir for regenerating capillary vessels.

Accordingly, the problem addressed by the present invention was to provide new active components which would satisfy the complex requirement profile described above. In addition, with the BSE debate in mind, this “multifunction component” would be a vegetable product.

### DESCRIPTION OF THE INVENTION

The present invention relates to cosmetic and/or pharmaceutical active components obtainable by fermenting plant constituents such as, for example, roots, nodules, leaves, fruit and/or plant extracts.

It has surprisingly been found that the fermentation products have a number of advantageous properties which are important for use in cosmetic products for the care and protection of skin and hair. Thus, the active components afford protection against the harmful effect of UV rays and show anti-inflammatory activity, for example, in cases of sunburn. They stimulate the metabolism in many ways. For example, they stimulate the synthesis of dermal macromolecules and, at the same time, inhibit their degradation. They improve hydration of the skin and the feeling of the skin.

The present invention also relates to a process for the production of cosmetic and/or pharmaceutical active components in which plant constituents and/or plant extracts are fermented. In this process,
(a) the plant constituents and/or plant extracts are size-reduced and/or pressed and/or extracted and processed to a fermentation broth,
(b) the fermentation broth is optionally pasteurized or sterilized,
(c) the fermentation broth thus prepared is inoculated with the microorganisms,
(d) the fermentation broth thus inoculated is fermented and, optionally,
(e) on completion of fermentation, the fermentation broth is worked up and the active components removed.

The plant constituents to be processed to a fermentation broth are selected from the group of seeds, nodules, roots, leaves, fruit, vegetable protein concentrates, isolates and/or hydrolyzates. Fruit and seeds are preferably used as the plant constituents.

Plant Constituents and/or Plant Extracts

Nodules, roots, leaves and preferably seeds and/or fruit are used in size-reduced and/or pressed and/or extracted form, preferably being selected from the group of plants and/or plant constituents of potatoes, rice, soya, wheat, barley, oats, rye, buckwheat, beans, peas, linseeds, cotton, sesame, lupins, rape, hemp, coconut palm, sunflowers, lucerne, hibiscus, maca, quinoa, almond, moringa, silk, baobao, cassia, irvinga, thistle and oil palm; or selected from the group of fruits consisting of apples, pears, quinces, medlars, rose hips, cherries, plums, peaches, apricots, pomegranates, berries, grapes, lemons, pineapples, cherimoya, guavas, mangos, starfruit, litchi, kiwi, banana, coconut, almonds, papaya, avocado, tamarind, baobao, sour sop, custard apple, mamay apple, atemoya, llama, sancoya, granadillo, sapodilla, rambutan, mangosteens, durian, bibasse (loquat), prickly pear cactus, pitahaya, langsat, jackfruit, chemdak, Virginian date plum, sharon fruit (kaki).

Microorganisms

In order to obtain an optimized yield of active components, it has proved to be of advantage to carry out the fermentation in the presence of a mixture of various microorganisms. A particularly preferred embodiment is characterized by the use of mixtures of various microorganisms which contain, on the one hand, at least one representative from the group consisting of Lactobacillus, Lactococcus and Leuconostoc and, on the other hand, at least one yeast. The following are typical examples of suitable microorganisms: Lactobacillus acidophilus, Lactobacillus brevis, Lactobacillus casei, Lactobacillus caucasicus, Lactobacillus cellobiosus, Lactobacillus delbruecki, Lactobacillus helveticus, Lactobacillus hilgardii, Lactobacillus kefir, Lactobacillus kefiranofaciens, Lactobacillus kefirgranum, Lactobacillus parakefir, Lactobacillus plantarum, Lactococcus lactis subsp. cremoris, Lactococcus lactis subsp. diacetilactis, Lactococcus lactis subsp.lactis, Lactococcus plantarum, Leuconostoc citreum, Leuconostoc citroverum, Leuconostoc dextranicum, Leuconostoc kefir, Leuconostoc mesenteroides, Leuconostoc pseudomesenteroides, Candida kefir, Candida tenuis, Kluyveromyces bulgaricus, Kluyveromyces fragilis, Kluyveromyces lactis, Saccharomyces carbajali, Saccharomyces carlbergensis, Sacharomyces cerevisiae, Saccharomyces delbrueckii, Saccharomyces florentinus, Saccharomyces globosus, Saccharomyces kefir, Saccharomyces marxianus, Saccharomyces, unisporus, Torula homii, Torula kefir, Streptococcus thermophilus, Streptococcus durans, Acetobacter aceti and Acetobacter rasens and mixtures thereof. Both the bacteria and the yeasts may be used in various ratios by weight, especially since these change during fermentation. The oculate of the lactic acid bacteria is between 10² and 10⁸ cfu/ml and preferably between 10³ and 10⁶ cfu/ml. The ratio of the lactic acid bacteria Lacotbacillus, Lactococcus and Leuconostoc to one another may be from 1:1000 to 1000:1 and is preferably from 1:100 to 100:1. Oculates of yeasts contain between 10² and 10⁷ cfu/ml and preferably between 10³ and 10⁵ cfu/ml. The ratio of lactic acid bacteria to yeasts is from 1:100000 to 100000:1 and preferably from 1:1000 to 1000:1. The microorganisms may be used in pure form although kefir or tibi mixtures commercially obtainable as such may also be used.

Fermentation

The fermentation process may be divided into five phases:
1. preparation of the fermentation broth/extraction,
2. optionally pasteurization or sterilization,
3. inoculation,
4. actual fermentation and optionally
5. working up of the products.
   Preparation of the Fermentation Both/Extraction

To prepare the fermentation broth, the vegetable starting materials (such as, for example, size-reduced plant parts, plant extracts, size-reduced and/or extracted seeds, nodules, roots or leaves, protein concentrates, hydrolyzates or isolates, size-reduced and/or extracted fruit)—depending on their hardness—are either directly ground or are first broken up and then further processed to an aqueous, organic or aqueous/organic dispersion. The extracts may be prepared in known manner, i.e. for example by aqueous, alcoholic or aqueous/alcoholic extraction of the plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Percolation is advantageous for industrial use.

Fresh plants or parts thereof are suitable as the starting material although dried plants and/or plant parts which may be mechanically size-reduced before extraction are normally used. Any size reduction methods known to the expert, for example freeze grinding, may be used. Preferred solvents for the extraction process are organic solvents, water or mixtures of organic solvents and water, more particularly low molecular weight alcohols with more or less high water contents. Extraction with methanol, ethanol, pentane, hexane, heptane, acetone, propylene glycols, polyethylene glycols, ethyl acetate and mixtures and water-containing mixtures thereof is particularly preferred. The extraction process is generally carried out at 20 to 100° C. and preferably at 30 to 60° C. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. Before inoculation with the microorganisms, the organic solvents are completely or substantially completely removed, for example by distillation or evaporation. If desired, the extracts thus prepared may be subjected, for example, to the selective removal of individual unwanted ingredients. The extraction process may be carried out to any degree, but is usually continued to exhaustion. Typical yields (=extract dry matter, based on the quantity of raw material used) in the extraction of seeds are in the range from 3 to 30 and more particularly 6 to 25% by weight.

The present invention includes the observation that the extraction conditions and the yields of the final extracts may be selected according to the desired application. These extracts generally have a solids content of 0.5 to 10% by weight. Suitable organic solvents in this connection are, for example, aliphatic alcohols containing 1 to 6 carbon atoms (for example ethanol), ketones (for example acetone), lower esters or polyols (for example glycerol or glycols).

The fermentation broth is generally prepared by—optionally repeated—extraction of the plant material with water in the mildly alkaline range, any insoluble solids being removed, for example, by filtration or centrifuging. In another advantageous embodiment of the invention, the extraction process is also carried out in aqueous medium, but in the acidic range, the proteins being precipitated, separated off and redissolved in water in the mildly alkaline range.

In another embodiment, the plant material is merely size-reduced, ground and dispersed in water or alkaline aqueous medium and directly fermented without further extraction or working up.

In another embodiment of the invention, the fermentation broth is prepared from commercially obtainable vegetable protein isolates or concentrates and dispersed in water or alkaline aqueous medium.

Where fruits are used as the starting material, they may either be ground or pressed so that the pulp or juice is used without further extraction.

To prepare the fermentation broth, other typical additives may be incorporated in these starting materials including, for example, soya peptone, malt extract or fermentable sugars (for example sucrose or glucose). It has proved to be of advantage to adjust the fermentation broths to a starting pH of 4.5 to 8.5 and, in the case of proteaginous educts, to a starting pH of 6.5 to 8.

Pasteurization or Sterilization

The pasteurization or sterilization of the fermentation broths is normally carried out over a period of 1 to 30 minutes at temperatures of 60 to 135° C.

Inoculation

The lactic acid bacteria and the yeasts may be used in different quanities and ratios by weight for the inoculation step. The bacteria are typically used in quantities of 10² to 10⁸ and preferably in quantities of 10³ to 10⁶ cfu/ml. The ratio by weight of the various lactic acid bacteria to one another, i.e. Lactobacillus, Lactococcus and Leuconcostoc, may be from 1:1000 to 1000:1 and is preferably from 1:100 to 100:1. The yeasts may be used in quantities of 10² to 10⁷ and preferably in quantities of 10³ to 10⁵ cfu/ml. The ratio by weight between bacteria and enzymes may ultimately be 1:100000 to 100000:1 and is preferably 1 :1000 to 1000:1.

Fermentation

The fermentation is normally carried out at temperatures of 10 to 47° C. and preferably at temperatures of 20 to 37° C. in a static or closed stirred tank. The fermentation time may vary between a few hours and a few days and is generally between 12 and 48 hours. In the course of the fermentation process, the fermentable sugars are converted into organic acids, ethanol, carbon dioxide and aromatics. Accordingly, there is a fall in the pH which generally settles at 4 to 5. In addition, the proteins present undergo proteolysis to form short-chain peptides and amino acids which are precipitated because of the acidic pH.

Working Up

Fermentation products which accumulate either as soluble fractions or as solid residues include the crude fermentation broth, the crude soluble fractions obtainable therefrom, the low molecular weight metabolites formed during fermentation (amino acids, oligopeptides, oligosaccharides, organic acids, aromatics, etc.), the solid residues of the fermented proteins precipitated and the fermented polysaccharides. These very different products can be recovered using separation techniques known per se such as, for example, centrifuging, membrane filtration (microfiltration, ultrafiltration, nanofiltration), liquid/liquid or solid phase extraction, chromatography, precipitation from solvents and the like. The microorganisms still present in the fermentation products must of course be removed, destroyed or inactivated before any making up into end products. This can be done by known techniques, such as heat treatment (pasteurization, sterilization), cell destruction, microfiltration, centrifuging and the like. If fermented products are obtained as end products, they may be used either as solid precipitates or—by lowering the pH—as solutions.

Cosmetic and/or Pharmaceutical Preparations

The present invention also relates to cosmetic and/or pharmaceutical preparations containing the new active components in quantities of preferably 0.01 to 5% by weight, more preferably 0.1 to 2% by weight and most preferably 0.5 to 1% by weight, based on the preparation. Before they are incorporated in cosmetic and/or pharmaceutical preparations, the fermentation products may optionally be encapsulated in microcapsules or nanocapsules by the usual methods.

The preparations according to the invention such as, for example, hair shampoos, hair lotions, foam baths, shower baths, creams, gels, lotions, alcoholic and aqueous/alcoholic solutions, emulsions, wax/fat compounds, stick preparations, powders or may contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, preservatives, perfume oils, dyes and the like as further auxiliaries and additives.

Surfactants

Suitable surfactants are anionic., nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicone, silicon methicone types, etc.) and/or aliphatic or naphthenic hydrocarbons such as, for example, squalane, squalene or dialkyl cyclohexanes.

Emulsifiers

Suitable emulsifiers are, for example, nonionic surfactants from at least one of the following groups:
products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group;
alkyl and/or alkenyl oligoglycosides containing 8 to 22 carbon atoms in the alk(en)yl group and ethoxylated analogs thereof;
addition products of 1 to 15 mol ethylene oxide onto castor oil and/or hydrogenated castor oil;
addition products of 15 to 60 mol ethylene oxide onto castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;
partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol,
mono-, di- and trialkyl phosphates and mono-, di- and/or tri-PEG-alkyl phosphates and salts thereof,
wool wax alcohols,
polysiloxane/polyalkyl/polyether copolymers and corresponding derivatives,
block copolymers, for example Polyethyleneglycol-30 Dipolyhydroxystearate;
polymer emulsifiers, for example Pemulen types (TR-1, TR-2) of Goodrich;
polyalkylene glycols and
glycerol carbonate.
   Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants.

Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminum and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-1 5, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol® 1789) or 1-phenyl-3-(4′-isopropylphenyl)-propane-1,3-dione and enamine compounds. The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminum and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, a-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prune extract, bambara nut extract, and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxy-benzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, lemon balm oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
non-aqueous solvents such as, for example, ethanol, propylene glycol and/or glycerol.

Suitable astringent active principles of antiperspirants are, above all, salts of aluminum, zirconium or zinc. Suitable antihydrotic agents of this type are, for example, aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachloro-hydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H )-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminum pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS (C.I. 69800) and madder lake (C.I. 58000). Luminol may also be present as a luminescent dye. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparations. The preparations may be produced by standard hot or cold processes and are preferably produced by the phase inversion temperature method.

Commercial Applications

The new active components have a number of properties which makes them interesting for the care and protection of skin and hair. Accordingly, the present invention also relates to their for the production of cosmetic or pharmaceutical preparations. Other advantageous embodiments of the invention relate to the use of the active components
for stimulating the growth and survival of fibroblasts;
for stimulating the GHS concentration in the cells;
as anti-inflammatory agents;
for protecting the skin and hair against UV-A radiation and UV-B radiation;
for protecting the DNA against damage by UV radiation;
for immunostimulation of the metabolism;
for combating wrinkles and for vitalizing and rejuvenating the skin;
for strengthening the defence mechanisms of skin and hair follicles against environmental toxins and oxidative stress;
for stimulating hair growth;
for stimulating fibroblasts to form dermal macromolecules, especially collagen;
for combating acne vulgaris;
as moisture regulators in the skin;
for cleansing the skin;
for inhibiting collagenases and elastases;
as regulators for melanogenesis in skin and hair and
as desliming agents.

### EXAMPLES

### Example 1

500 g pea seeds were size-reduced, dispersed in 10 times the quantity of water and the resulting dispersion adjusted to pH 4.7 by addition of sulfuric acid. The suspension was then stirred for 2 h at 52° C., cooled and the undissolved constituents removed by centrifuging. 0.36 g of the residue precipitated in the acid range (pea acid precipitate) were suspended in 750 ml water and, after stirring for 45 minutes, the pH was raised in stages to 7.5 by addition of sodium hydroxide. The undissolved constituents were again removed. The soluble extract (0.8 kg) was transferred to a fermentation tank in which it was incubated for 20 mins. at 90° C. The preparation was then cooled to 20° C. and 0.2% w/v of the commercially obtainable Wiesby culture C1 containing the following microorganisms was added: Lactococcus lactis, Lactococcus cremoris, Lactococcus deacetylactis, Leuconostoc, Lactobacillus kefyr, Candida kefyr, Saccharomyces kefyr. Fermentation was carried out in a closed tank at 22° C. at a stirring speed of 100 r.p.m. After a fermentation time of 27.5 h, the pH had fallen to 4.5. The fermentation broth was centrifuged, the supernatant solution was incubated for 20 mins. at 90° C., cooled, concentrated under reduced pressure and then freeze-dried. The yield amounted to 7% by weight, based on the starting materials (g/g dry weight of the pea acid precipitate). The end product had a nitrogen content of 5% by weight.

### Example 2

The procedure was as in Example 1, except that fermentation was carried out with 800 g pea extract, 0.8 g yeast extract and 4 g sodium chloride. After freeze-drying, the yield of fermentation products was 9% by weight, based on the starting materials (g/g dry weight of the pea acid precipitate). The end product had a nitrogen content of 4% by weight.

### Example 3

Example 2 was repeated, After centrifuging of the fermentation broth, the fermented protein fraction was removed and resuspended in 5 times the volume of water. The suspension was stirred for 30 mins. and, at the same time, the pH value was adjusted in stages to 7.6 by addition of sodium hydroxide. The suspension was then incubated for 20 mins. at 90° C. and freeze-dried. The fermentation product was obtained in a yield of 8% by weight, based on the starting materials (g/g dry weight of the pea acid precipitate) and had a nitrogen content of 12% by weight.

### Example 4

5.4 g ground Hibiscus esculentus seeds were dispersed in 30 kg water at 50° C. and 0.61 kg NaOH pellets were added to the resulting dispersion. The suspension was stirred for 4 hours at 50° C., cooled and centrifuged. The 23.4 kg supernatant solution obtained were adjusted to pH 7.8 by addition of sulfuric acid and then spray-dried. 900 g hibiscus extract were obtained. 16 g of the solid powder were transferred to a fermenter together with 0.8 g yeast extract and 4 g sodium chloride and, after the addition of 800 ml water (pH 7.8), were incubated for 15 mins. at 121° C. The fermentation broth was cooled to 22° C. and 0.1% w/v of the culture Kefir C1 was added. The extract was fermented in a closed tank for 2 days at 22° C. and at a stirring speed of 100 r.p.m. (pH 4.9), incubated for 20 mins. at 80° C. and centrifuged. The supernatant solution was then concentrated under reduced pressure and freeze-dried. The fermentation product was obtained in a yield of 60% by weight, based on the dry matter of the fermentation broth. The resulting product had a nitrogen content of 3.5% by weight.

### Example 5

Fruits of the palm Bactris were broken up in the presence of water, resulting in the formation of a suspension with a solids content of 10% by weight. The suspension was incubated for 30 mins. at 110° C. and cooled to 30° C. The commercially available ferment Kefir Fruit (Yalacta) containing the following microorganisms was then added: Lactococcus lactis, Lactococcus cremoris, Lactococcus deacetylactis, Leuconostoc, Lactobacillus caucasicus, Lactococcus lactis subsp. lactis and Saccharomyces florentinus. 4 g of the ferment were suspended in 20 ml of a sterile 0.9% by weight sodium chloride solution and 2% w/v of the solution were added to the palm fruit fermentation broth. The broth was fermented in a closed tank at 30° C. and at a stirring speed of 150 r.p.m. After 24 h, the pH had fallen to 5.0. The fermentation broth was incubated for 30 mins. at 90° C., cooled, filtered through a 500 μm Nylon sieve, washed with 150 ml water and then centrifuged. The residue was again washed with 200 g water and the suspension was centrifuged. The supernatant solutions were combined, concentrated under reduced pressure and finally freeze-dried. The fermentation product was obtained in a yield of 45% by weight, based on the dry weight of the fermentation broth.

### Example 6

Example 5 was repeated. The fermentation broth was prepared with a starting content of 5% g/g dry weight fruit of the palm Bactris and 1.25% by weight glucose and 0.04% by weight malt extract. The fermentation product was obtained in a yield of 45% by weight, based on the dry weight of the fermentation broth.

### Example 7

1 kg maca root powder (Amazonian Natural Product, Peru) was dispersed in distilled water so that a 10% by weight dispersion was obtained. The pH was adjusted to a value of 7-7.2 with a 4 N sodium hydroxide solution. The suspension was stirred for one hour at room temperature (22° C.±2° C.) and then incubated with 0.1% (w/v) of a commercial kefir culture (Wiesby Kefir C1). The maca broth was fermented at room temperature (20-25° C.) and at a stirring speed of 100 r.p.m. After 1.5 days (pH 4), the broth was heated for 15 minutes to 70-80° C. and then centrifuged and filtered to remove insoluble constituents. The solution thus obtained was freeze-dried. A fermentation product was obtained in a yield of 28% by weight, based on the dry weight of the maca powder, and had a nitrogen content of 2.6% by weight.

### Example 8

5 g quinoa seeds were size reduced and dispersed in distilled water to form a 10% by weight dispersion. The pH of the dispersion was adjusted to a value of 7-7.2 with a 4 N sodium hydroxide solution. The suspension was stirred for 1 hour at room temperature (22° C.±2° C.) and then incubated with 1% (w/v) of a commercial kefir culture (Wiesby Kefir C1). The quinoa broth was fermented at room temperature (22° C.±2° C.) and at a stirring speed of 100 r.p.m. (pre-culture). After 24 hours (pH 4.2), the fermentation broth (pre-culture) was used to incubate 5 kg suspension from 500 g size-reduced quinoa as previously prepared. The quinoa broth was fermented at room temperature (22° C.±2° C.) and at a stirring speed of 300 r.p.m. After 28 hours (pH 4.3), the broth was heated for 15 mins. to 70-80° C. and then centrifuged and filtered to remove insoluble constituents. The solution thus obtained was freeze-dried. A fermentation product was obtained in a yield of 11% by weight, based on the dry weight of the quinoa powder, and had a nitrogen content of 6.9% by weight.

Regenerative and Growth-Stimulating Activity

After incubation for 72 h in a nutrient solution, fibroblasts form saturated monolayers, the fibroblasts cease their activity and growth stops. The cell fuel adenosine triphosphate (ATP), which is essentially formed in the mitochondria, is needed to activate certain enzymes which, for example, control the cell skeleton, the ionic channels, the uptake of nutrients and a large number of other important biological processes. The protein content of the cells was determined by Bradford's method [cf. Anal. Biochem. 72, 248-254 (1977)]. Glutathione (GSH) is a special protein which is produced by the cells for protection against oxidative stress and environmental poisons, more particularly against heavy metals. The three amino acids involved in the reduced form of GSH are linked to special cytoplasmatic enzymes which need ATP for activation. An increase in the GSH concentration leads to an increase in the glutatione-S-transferase activity, a detoxifying enzyme. The GHS content was determined by Hissin's method [cf. Anal. Biochem. 74, 214-226 (1977]. The growth-stimulating effect of the test substances was tested on human fibroblasts. In a first series of tests, the fibroblasts were incubated in a nutrient medium for 1 day at 37° C./5% by vol. CO₂, the nutrient medium was replaced by a medium which contained the test substance and the fibroblasts were incubated for another 3 days at 37° C. The protein content of the cells and the ATP concentration were then determined. The survival-stimulating effect was determined in a second series of tests. To this end, the fibroblasts were incubated first for 3 days at 37° C. in a nutrient solution and then for 3 days at the same temperature in a test solution. The protein content of the cells and the GSH concentration were then determined. The number of living cells was determined in a few tests by measuring the content of cellular ATP and cellular DNA. The results are set out in Table 1. in %-rel. against a blank sample and represent the results of 3 series of measurements involving triple determination.

The results show that the test substances stimulate the metabolism in regard to growth and protection of the fibroblasts.

TABLE 1
Growth- and survival-stimulating effect (figures = % -rel.)

| | Conc. | | | Test series 2 (T 2) | | | |
|---|---|---|---|---|---|---|---|
| | % w/v | Test series 1 (T 1) | | | GSH/ | | |
| Extract | (T1/T2) | Proteins | ATP | Proteins | protein ratio | ATP | DNA |
| Blank | 0 | 100 | 100 | 100 | 100 | 100 | 100 |
| sample | | | | | | | |
| Ex. 1 | 0.1/0.1 | | | 134 ± 0 | 169 ± 0 | | |
| Ex. 2 | 0.1/0.1 | 153 ± 10 | 115 ± 6 | 145 ± 15 | 143 ± 1 | | |
| Ex. 3 | 0.1/0.1 | 151 ± 11 | 119 ± 8 | 136 ± 10 | 134 ± 1 | | |
| Ex. 4 | 0.1/0.02 | 119 ± 2 | | 131 ± 8 | | | |
| Ex. 5 | 0.1/0.1 | 111 ± 32 | 145 ± 32 | | | | |
| Ex. 6 | 0.1/0.1 | 155 ± 14 | 121 ± 20 | 163 ± 4 | 111 ± 6 | | |
| Ex. 7 | 0.1/0.3 | 137 ± 3 | | 169 ± 13 | | 186 ± 1 | 145 ± 1 |
| Ex. 8 | 0.1/0.3 | 121 ± 12 | | 116 ± 23 | | 110 ± 11 | 112 ± 6 |

Anti-Inflammatory Activity

In the course of cutaneous inflammation, leucocytes, such as the polymorphonuclear neutrophilic granulocytes (PMNs) for example, are stimulated by peptides, such as cytokinins for example, to emit messenger substances, such as leucotriene for example, which are released from activated or necrotic cells in the dermis. These activated PMNs release not only pro-inflammatory cytokinins, leucotrienes and proteases, but also ROS, such as superoxides and hypochlorite anions for example, of which the function is to destroy penetrated pathogenic germs or fungi. This activity of the PMNs during the inflammation is known as so-called respiratory burst and can lead to additional damage in the tissue. To investigate to what extent the test extracts can prevent or reduce the respiratory burst, a cell line of human leukaemic granulocytes of these PMNs was incubated together with the test substances at 37° C. and 5% by vol. CO₂. After the respiratory burst had been initiated by addition of a yeast extract (zymosan) to the cell solution, the release of superoxide anions was determined through their reaction with luminol. The results are set out in Table 2 which shows the cell counts and the quantity of ROS released in %-rel to the standard as the mean value of a series of measurements involving triple determination.

The results show that the test substances have a strong inhibiting influence on the respiratory burst of human granulocytes but do not damage the granulocytes.

TABLE 2
Anti-inflammatory activity

| | Conc. | | |
|---|---|---|---|
| Extract | % by weight | Cell counts | ROS released |
| Control | | 100 | 100 |
| Ex. 1 | 0.1 | 95 ± 3 | 29 ± 8 |
| Ex. 2 | 0.1 | 99 ± 2 | 42 ± 9 |
| Ex. 4 | 0.1 | 94 ± 3 | 38 ± 13 |
| Ex. 5 | 0.1 | 102 ± 5 | 54 ± 15 |
| Ex. 6 | 9.1 | 97 ± 5 | 53 ± 3 |

Protecting Cells Against UVB Radiation

The function of this test was to show that the test substances have anti-inflammatory properties for human keratinocytes. UVB was selected as the stress factor because the rays produce cutaneous inflammation (erythemas, oedemas) by activating enzymes that release arachidonic acid, such as phospholipase A2 (PLA2) for example. This results not only in damage to the membranes, but also in the formation of inflammatory substances, such as prostaglandins of the PGE2 type for example. The influence of UVB rays on keratinocytes was determined in vitro through the release of cytoplasmatic enzymes, such as LDH (lactate dehydrogenase) for example, which runs parallel to the cell damage and the formation of PGE2. To carry out the test, a fibroblast culture was mixed with foetal calf serum and inoculated with the test substances 2 days later. After incubation for 36 h at 37° C. and a CO₂ level of 5% by vol., the nutrient medium was replaced by an electrolyte solution and the fibroblasts were damaged with a particular dose of UVB (50 mJ/cm²). The quantity of keratinocytes was determined after trypsination via a cell counter while the LDH concentration was enzymatically determined and the PGE2 formed was measured by Elisa Test. The results are set out in Table 3 which shows the activity in %-rel. against a standard as the mean value of two test series involving double determination.

TABLE 3
Effect against UVB rays (figures - %-rel.)

| | Conc. | Keratinocyte | LDH | PGE2 |
|---|---|---|---|---|
| Extract | % w/v | count | released | released |
| Control without UVB | | 100 | 0 | 0 |
| Control with UVB | | 24 ± 5 | 100 | 100 |
| Ex. 1 + UVB | 0.03 | 74 ± 4 | 24 ± 11 | |
| Ex. 2 + UVB | 0.3 | 136 ± 6 | 0 ± 2 | |
| Ex. 3 + UVB | 0.1 | 170 ± 14 | 10 ± 2 | |
| Ex. 7 + UVB | 0.3 | 44 ± 0 | 22 ± 7 | 26 ± 9 |
| Ex. 8 + UVB | 0.3 | 141 ± 22 | 0 ± 2 | 0 ± 1 |

The results show that the test substances significantly reduce the harmful effects of UVB rays and, in particular, reduce the release of LDH and PGE2.

Cytophotoprotection of Human Fibroblasts

The protection of cells against UV-A radiation was evaluated by a test on human fibroblasts because UV-A radiation penetrates through the epidermis and causes damage by oxidative stress in the region of the dermis (DALLE CARBONARE, M., PATHAK, M.A.: Skin photosensitizing agents and the role of reactive oxygen species in photoaging; JOURNAL OF PHOTOCHEMISTRY & PHOTOBIOLOGY, 1992, 14, 1-2, 105-124 (P 10482). The level of oxidative stress was determined in vitro by determining the content of malondialdehyde released and intracellular GSH (reduced glutathione) (Morlière, P., Moisan, A., Santus, R., Huppe, G., Mazière, J. C., Dubertret, L.: UV-A induced lipid peroxydation in cultured human fibroblasts, Biochim. Biophys. Acta, 1084, 3:261-269 (1991).

Method:

Inoculation of human fibroblasts in nutrient medium (standard medium containing foetal calf serum (FCS), incubation for 3 days at 37° C./5% CO₂. The nutrient medium was replaced by a standard medium with no FCS, but with active component, and re-incubated for 3 days at 37° C./5% CO₂. The nutrient medium was then replaced by isotonic salt solution and the fibroblasts were exposed to UV-A radiation of 20 J/cm₂ (black light TFWN lamp). The content of malondialdehyde (MDA level) in the supernatant medium was then determined by spectrophotometry. The number of cells was measured by the Bradford method via the content of cell proteins. The results are set out in Table 4 in % versus the control (without exposure to UV) and represent the mean values of two determinations carried out three times.

TABLE 4
Cytophotoprotection against UV-A radiation on human fibroblasts

| | | MDA released | Cell protein |
|---|---|---|---|
| | % w/v | Mean value | Mean value |
| Control without UV | | 0 | 100 |
| UVA 20 Jcm² | | 100 | 114 |
| Ex. 7 + UVA | 0.01 | 68 | 136 |
| Ex. 8 + UVA | 0.3 | 42 | 187 |

The results show that the fermentation products tested lead to a significant reduction in the damage caused by UV-A radiation. Accordingly, the fermentation products are advantageous for improving the resistance of skin and hair follicles to oxidative stress applied by UV radiation and environmental poisons. They protect skin and hair follicles against ageing.

Inhibition of Elastase

Elastase is a protease secreted by leucocytes in the event of inflammation or by fibroblasts after exposure to UV radiation or by ageing. It is an enzyme which catalyzes the destruction of key dermal proteins such as, for example, proteoglycans, elastin or collagen fibers and thus induces the intrinsic ageing and also the photo-ageing of human skin (ROBERT, L., LABAT ROBERT, J.: Vieillissement et tissu conjonctif. Année Gérontologique, 23-27,1992).

Method:

Test method of BIETH, J.: Elastase: Structure, Function and Pathological Role, Front Matrix Biol., 6:1-82, Karger Basel, 1978.

The test was carried out with elastase from pancreas colored with Congo Red. The incubation time at room temperature was 30 minutes and the optical density of the Congo Red released was determined after centrifuging at a wavelength of 520 nm.

The results are set out in Table 5 as % inhibition versus a control (=0%).

TABLE 5
Elastase inhibition in tubo

| | % w/v | Elastase inhibition |
|---|---|---|
| Control | 0% | 0 |
| Ex. 3 | 0.3% | 38 |

The fermentation product tested showed good inhibition of the release of elastase and may therefore be successfully used against ageing of the skin and damage by UV radiation.

Modulation of Melanogenesis

The influence on melanogenesis was determined by a test with an in vitro culture of B16 melanocytes.

Method:

Melanocytes (B16 cell line) were incubated in standard growth medium containing foetal calf serum (FCS) for 3 days at 37° C./5% CO₂. The growth medium was replaced by standard medium containing different concentrations of fermentation product. After further incubation for 3 days, the number of living cells was determined by counting cell proteins by the Bradford method and the content of synthesized melanin was detected by measuring the optical density at 475 nm in the cell homogenizate.

The results are set out in Table 6 as % versus a control (cell culture with no fermentation product).

TABLE 6
Melanogenesis test
Results in %/control: (mean value of 2 or 3 assays (± SEM))

| | % w/v | Cell proteins | Melanin level |
|---|---|---|---|
| Control | 0 | 100 | 100 |
| Ex. 8 | 0.1 | 112 ± 7 | 161 ± 17 |
| Ex. 8 | 0.3 | 108 ± 6 | 181 ± 54 |
| Ex. 8 | 1 | 188 ± 8 | 51 ± 36 |

The fermented product tested showed a high potential for modulating the synthesis of melanin in in-vitro cultures of melanocytes.

Immunostimulation

Immunostimulation is the umbrella term for biochemical processes in which messenger substances, such as β-glucans for example, stimulate the body's own defences, for example for binding and secreting toxins and accelerating the renewal of skin cells. It is known that organisms lose this ability with increasing age. Immunostimulation can be observed in vitro on human leucocytes activated beforehand with a yeast extract (zymosan) [cf. Capsoni et al., mnt. J. Immunopharm. 10(2), 121-133 (1998)]. A culture of polymorphonuclear neutrophilic granulocytes (PMNs) was incubated with the test substances for 24 h at 37° C./5% by vol. CO₂. The addition of zymosan initiated the respiratory burst. After 30 mins, the PMN count was determined with an automatic cell counter while the quantity of reactive oxygen species (ROS) released in the supernatant liquid was spectroscopically determined with luminol. The results are set out in Table 4 as %-rel against the standard. Table 4 shows the mean value of two series of measurements involving triple determination.

TABLE 4
Immunostimulation (figures in % rel.)

| | Conc. | No. of | |
|---|---|---|---|
| Ex. | % w/v | leucocytes | ROS released |
| Blank sample | 0 | 100 | 100 |
| Ex. 3 | 0.01 | 99 ± 3 | 165 ± 7 |

The results show that the test substances stimulate the immune system and lastingly strengthen the body's own defences, more particularly the skin cells.

Ex-Vivo Determination of the Moisturizing Effect

The dry Stratum corneum is a dielectric medium with weak conductivity. If moisture is supplied to the Stratum corneum, its conductivity increases due to the bipolar character of the water molecules. Accordingly, conductometry is a suitable method for determining the hydration status of the Stratum corneum. If conductivity is improved by the addition of test substances, it may be concluded that these substances have a moisturizing effect. The tests were carried out using an in-vitro skin model which had been prepared beforehand by the Obata and Tagami method described in J. Soc. Cosmet. Chem., 41, 235-242 (1990). The preparations were equilibrated in chambers of defined air humidity and then tested under three or four different conditions:
control test with no treatment
blank test with placebo treatment
test with test substance according to the invention
comparison test with standard preparation

The conductivity measurements were carried out before the treatment and then over a period of 0.5 to 24 after the treatment. The results are set out in Table 5.

TABLE 5
Hydration measurements

| | Conductivity [μS] after h | | | | | | |
|---|---|---|---|---|---|---|---|
| Extract | Before | 0.5 | 1 | 2 | 4 | 6 | 24 |
| Test Series 1 | | | | | | | |
| Control test | 27.7 | 30.2 | 32.9 | 30.0 | 33.1 | 29.2 | 32.7 |
| Placebo emulsion | 33.4 | 36.2 | 39.2 | 44.1 | 39.4 | 38.3 | 38.4 |
| 1.5% w/v Ex. 4 | 36.9 | 95.2 | 66.9 | 55.6 | 53.7 | 54.3 | 48.2 |
| 1.5% w/v glycerin | 33.6 | 62.7 | 64.7 | 53.6 | 46.9 | 52.0 | 55.1 |
| Test Series 2 | | | | | | | |
| Control test | 24.5 | 26.2 | 28.3 | 28.2 | 25.3 | 25.3 | 23.1 |
| Placebo emulsion | 17.8 | 39.0 | 33.5 | 29.1 | 26.5 | 26.9 | 28.8 |
| 1.5% w/v Ex. 2 | 22.9 | 64.5 | 48.1 | 44.9 | 45.3 | 42.0 | 35.4 |

The Examples show that the hydration status of the Stratum corneum is significantly improved, even by comparison with known moisturizers, by addition of the test substances. A cream containing 1.5% by weight of the extract of Example 4 produced an improvement in hydration of more 160% after only 30 mins. whereas a comparison cream containing the same quantity by weight of glycerin produced an improvement of only about 75%.

In-Vivo Measurement of the Moisturizing Effect

Similarly to the ex-vivo determination, the hydration status of the skin can also be determined in vivo by conductometry within the framework of a non-invasive measurement. To this end, conductivity is determined over an area of 4 cm² on the inside of the forearm without any treatment with the test substance (T0 value). 4 μl/cm² of test substance is then applied and dried for 15 mins., after which conductivity is re-measured (T15 value). In addition, for control purposes, the conductivity of an adjacent area of skin that had not been treated with the test substance was measured. The results are expressed as the percentage T15:T0 ratio and are set out in Table 6.

TABLE 6
Hydration measurements [% -rel.]

| | Improvement of |
|---|---|
| Extract | hydration T15/T0 |
| Ex. 1 | 7.3 |
| Ex. 2 | 9.3 |
| Ex. 4 | 19.6 |

The results show that the test substances significantly improve the hydration status of the skin.

Improvement of Skin Roughness

The influence the test substances exert on the roughness or softness of the skin can be determined in vivo by frictiometry. The principle of this method consists in the application of a constant pressure to the surface of the skin by a body of rotation. A measurement is made of the necessary force through which the friction coefficient can be determined. The force which has to be applied is directly dependent on the roughness of the skin. It follows from this that the coefficient is greater, the higher the hydration status of the skin, i.e. the softness. To this end, friction is first determined without treatment with the test substance over an area of 9 cm² on the inside of the forearm (T0 value). 4 μl/cm² of test substance is then applied and dried for 15 mins., after which friction is re-measured (T15 value). In addition, for control purposes, the conductivity of an adjacent area of skin that had not been treated with the test substance was measured. The results are expressed as the percentage T15:T0 ratio and are set out in Table 7.

TABLE 7
Friction measurements [% -rel.]

| | Improvement of |
|---|---|
| Extract | skin roughness T15/T0 |
| Ex. 1 | 6 |
| Ex. 2 | 73 |
| Ex. 4 | 28 |
| Ex. 6 | 130 |

The results show that the test substances significantly improve the roughness of the skin and make it much softer.

A number of Formulation Examples are set out in Table 8 below.

TABLE 8
Examples for cosmetic preparations (water, preservative to 100% by weight)

| Composition (INCI) | 1 | 2 | 3 | 4 | 5 | 6 | 7 | 8 | 9 | 10 |
|---|---|---|---|---|---|---|---|---|---|---|
| Emulgade ® SE | 5.0 | 5.0 | 4.0 | — | — | 5.0 | 5.0 | 4.0 | — | — |
| Glyceryl Sterate (and) Ceteareth 12/20 (and) Cetearyl | | | | | | | | | | |
| Alcohol (and) Cetyl Palmitate | | | | | | | | | | |
| Eumulgin ® B1 | — | — | 1.0 | — | — | — | — | 1.0 | — | — |
| Ceteareth-12 | | | | | | | | | | |
| Lameform ® TGI | — | — | — | 4.0 | — | — | — | — | 4.0 | — |
| Polyglyceryl-3 Isostearate | | | | | | | | | | |
| Dehymuls ® PGPH | — | — | — | — | 4.0 | — | — | — | — | 4.0 |
| Polyglyceryl-2 Dipolyhydroxystearate | | | | | | | | | | |
| Monomuls ® 90-O 18 | — | — | — | 2.0 | — | — | — | — | 2.0 | — |
| Glyceryl Oleate | | | | | | | | | | |
| Cetiol ® HE | — | — | — | — | 2.0 | — | — | — | — | 2.0 |
| PEG-7 Glyceryl Cocoate | | | | | | | | | | |
| Cetiol ® OE | — | — | — | 5.0 | 6.0 | — | — | — | 5.0 | 6.0 |
| Dicaprylyl Ether | | | | | | | | | | |
| Cetiol ® PGL | — | — | 3.0 | 10.0 | 9.0 | — | — | 3.0 | 10.0 | 9.0 |
| Hexyldecanol (and) Hexyldecyl Laurate | | | | | | | | | | |
| Cetiol ® SN | 3.0 | 3.0 | — | — | — | 3.0 | 3.0 | — | — | — |
| Cetearyl Isononanoate | | | | | | | | | | |
| Cetiol ® V | 3.0 | 3.0 | — | — | — | 3.0 | 3.0 | — | — | — |
| Decyl Oleate | | | | | | | | | | |
| Myritol ® 318 | — | — | 3.0 | 5.0 | 5.0 | — | — | 3.0 | 5.0 | 5.0 |
| Coco Caprylate Caprate | | | | | | | | | | |
| Bees Wax | — | — | — | 7.0 | 5.0 | — | — | — | 7.0 | 5.0 |
| Nutrilan ® Elastin E20 | 2.0 | — | — | — | — | 2.0 | — | — | — | — |
| Hydrolyzed Elastin | | | | | | | | | | |
| Nutrilan ® I-50 | — | 2.0 | — | — | — | — | 2.0 | — | — | — |
| Hydrolyzed Collagen | | | | | | | | | | |
| Gluadin ® AGP | — | — | 0.5 | — | — | — | — | 0.5 | — | — |
| Hydrolyzed Wheat Gluten | | | | | | | | | | |
| Gluadin ® WK | — | — | — | 0.5 | 0.5 | — | — | — | 0.5 | 0.5 |
| Sodium Cocoyl Hydrolyzed Wheat Protein | | | | | | | | | | |
| Extract of Example 2 | 1.0 | 1.0 | 1.0 | 1.0 | 1.0 | — | — | — | — | — |
| Extract of Example 6 | — | — | — | — | — | 1.0 | 1.0 | 1.0 | 1.0 | 1.0 |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Magnesium Sulfate Hepta Hydrate | — | — | — | 1.0 | 1.0 | — | — | — | 1.0 | 1.0 |
| Glycerin (86% by weight) | 3.0 | 3.0 | 5.0 | 5.0 | 3.0 | 3.0 | 3.0 | 5.0 | 5.0 | 3.0 |

(1, 6) Soft cream,
(2, 3, 7, 8) Moisturizing emulsion,
(4, 5, 9, 10) Night cream

1-40. (canceled)

41. A process for producing a cosmetic and/or pharmaceutical active component, said process comprising:
(a) providing a fermentation broth comprising a plant component selected from the group consisting of plant constituents, plant extracts and mixtures thereof;
(b) inoculating the fermentation broth with a microorganism; and
(c) fermenting the microorganism-containing fermentation broth to produce an active component.

42. The process according to claim 41, wherein the plant component is derived from a plant selected from the group consisting of potatoes, rice, soya, wheat, barley, oats, rye, buckwheat, beans, peas, linseeds, cotton, sesame, lupins, rape, hemp, coconut palm, sunflowers, lucerne, hibiscus, maca, quinoa, almond, moringa, silk, baobao, cassia, irvinga, thistle, oil palm and mixtures thereof.

43. The process according to claim 41, wherein the plant component comprises a plant constituent selected from the group consisting of seeds, nodules, roots, leaves, fruits and mixtures thereof.

44. The process according to claim 41, wherein the plant component is selected from the group consisting of protein concentrates, hydrolyzates and isolates.

45. The process according to claim 41, wherein the fermentation broth is adjusted to a pH of from 4.5 to 8.5 prior to fermentation.

46. The process according to claim 41, further comprising pretreating the fermentation broth prior to inoculating, wherein the pretreatment is selected from pastuerization, sterilization and combinations thereof.

47. The process according to claim 46, wherein the pretreatment is carried out at a temperature of from 60 to 135° C.

48. The process according to claim 46, wherein the pretreatment is carried out over a period of from 1 to 30 minutes.

49. The process according to claim 41, wherein the fermentation broth is inoculated with a mixture of microorganisms.

50. The process according to claim 41, wherein the microorganism comprises a component selected from the group consisting of Lactobacillus, Lactococcus and Leuconostoc.

51. The process according to claim 49, wherein at least one microorganism comprises a component selected from the group consisting of Lactobacillus, Lactococcus and Leuconostoc.

52. The process according to claim 51, wherein at least one microorganism comprises a yeast.

53. The process according to claim 41, wherein the fermentation is carried out at a temperature of from 10 to 47° C.

54. The process according to claim 41, further comprising separating the active component from the fermented broth, wherein the separation is carried out in a manner selected from the group consisting of centrifugation, filtration, extraction, chromatography and precipitation.

55. The process according to claim 41, wherein the plant component is derived from a pea plant.

56. A process for producing a cosmetic and/or pharmaceutical active component, said process comprising:
(a) providing a fermentation broth comprising a plant component selected from the group consisting of plant constituents, plant extracts and mixtures thereof, and subjecting the fermentation broth to a pretreatment selected from pastuerization, sterilization and combinations thereof;
(b) inoculating the fermentation broth with a mixture of microorganisms, wherein at least one microorganism comprises a component selected from the group consisting of Lactobacillus, Lactococcus and Leuconostoc, and wherein at least one microorganism comprises a yeast;
(c) fermenting the microorganism-containing fermentation broth to produce an active component; and
(d) separating the active component from the fermented broth, wherein the separation is carried out in a manner selected from the group consisting of centrifugation, filtration, extraction, chromatography and precipitation.

57. A cosmetic and/or pharmaceutical preparation comprising an active component prepared by the process according to claim 41.

58. A cosmetic and/or pharmaceutical preparation comprising an active component prepared by the process according to claim 56.

59. The preparation according to claim 57, wherein the active component is present in an amount of from 0.01 to 5% by weight, based on the preparation.

60. A method of treating a substrate, said method comprising:
(a) providing a substrate selected from the group consisting of skin and hair; and
(b) contacting the substrate with an active component prepared by the process according to claim 41.
